(19) United States
(12) Patent Application Publication
(10) Pub. No.: US 2005/0238724 A1
(43) Pub. Date: Oct. 27, 2005

(54) Pharmaceutical composition containing lamotrigine particles of defined morphology

(75) Inventors: Judith Aronhime, Rehovot (IL); Guy Samburski, Ganot-Hadar (IL)

Correspondence Address: KENYON & KENYON, ONE BROADWAY, NEW YORK, NY, 10004, US

(73) Assignee: Teva Pharmaceutical Industries Ltd., Petah Tiqva (IL)

(21) Appl. No.: 10/511,987

(22) PCT Filed: Apr. 23, 2003
(86) PCT No.: PCT/US03/13002

§ 371 Date: Oct. 21, 2004

Related U.S. Application Data
(60) Provisional application No. 60/374,923, filed on Apr. 23, 2002.

(51) Int. Cl. A61K 31/53; A61K 9/14

(52) U.S. Cl. 424/489; 514/242

(57) ABSTRACT

The present invention provides a pharmaceutical composition comprising a plurality of lamotrigine particles having a specific surface area of from about two to about three and a half meters per gram. Pharmaceutical compositions falling within the surface area criteria for the lamotrigine particles include those having a particle diameter equal to or less than about 100 μm, preferably about 50 μm, and most preferably 10 μm. The pharmaceutical composition can be formulated into a wide variety of dosage forms.

### CROSS-REFERENCE TO RELATED APPLICATION

This application claims the benefit of U.S. provisional application No. 60/374,923 filed on Apr. 23, 2002, the disclosure of which is entirely incorporated by reference.

### FIELD OF THE INVENTION

The present invention relates to anti-seizure drugs, more particularly to lamotrigine of defined morphology which is used as an adjunct medication in epilepsy therapy, and to methods of preparing pharmaceutical compositions containing such lamotrigine.

### BACKGROUND OF THE INVENTION

Lamotrigine, whose systematic chemical name is 3,5-diamino-6-(2,3-dichlorophenyl)-1,2,4-triazine, exhibits anticonvulsive activity in humans who are prone to seizures. It is approved by the U.S. Food and Drug Administration for use as an adjunct medication in epilepsy therapy.

Lamotrigine is reported to be poorly soluble in water (0.17 mg ml⁻¹) at room temperature and its solubility is not significantly enhanced by changes in pH.

Development of parenteral formulations of lamotrigine has been hampered by its low aqueous solubility. A sterile aqueous solution of a drug is a desirable parenteral administration vehicle, but due to lamotrigine's low solubility, parenteral administration in such a vehicle would necessitate injecting an undesirably large volume of solution. Pharmaceutical formulators have attempted to address use limitations caused by lamotrigine's poor solubility by developing a water-soluble salt of lamotrigine. Unfortunately, salts of lamotrigine with most widely used pharmaceutically benign counterions, such as citrate, tartrate, or maleate, also have low solubility in aqueous solutions.

U.S. Pat. No. 4,847,249 discloses a soluble lamotrigine salt with 2-hydroxyethanesulfonic acid (isethionic acid). Unfortunately, isethionic acid is reported to be unstable to prolonged storage. Consequently, it must be generated, e.g. from a stable metal salt, and used quickly to prepare the lamotrigine salt. Problems with the lability of the counter-ion when in solution with tonicity modifiers customarily employed in parenteral formulations further make this salt an inconvenient form in which to administer the drug.

U.S. Pat. No. 5,942,510 (the '510 patent) discloses a lyophilized formulation of lamotrigine mesylate and a bulking agent that can be reconstituted with a liquid carrier up to a lamotrigine concentration of 60 mg ml⁻¹. The reconstituted solution is acidic, having a pH from 2.5 to 5. Parenteral administration of acidic solutions can be painful for the patient. However, the '510 patent warns that a pH buffering agent should not be added to either the freeze-dried formulation or the injectable solution.

Aside from the absence of a counterion that significantly improves the solubility of lamotrigine while allowing it to be administered conveniently and without distress to the patient, lamotrigine is not a good candidate for administration as an acid addition salt because it is reported to be acid sensitive.

Physical as well as chemical modifications can affect a compound's solubility properties, such as the crystalline or amorphous state of the compound and particle size. “Solubility” is the concentration of a solution phase at equilibrium with a given solid phase of the dissolved substance. In a compound that is capable of adopting two or more solid phases (crystalline modifications), a more highly soluble crystalline modification of a compound is thermodynamically less stable than a less soluble crystal modification under those same conditions. Thus, providing a thermodynamically less stable modification may be expected to yield a more highly concentrated solution of the compound in equilibrium with the solid phase compound (although kinetic factors may slow the attainment of equilibrium and the less soluble modification may precipitate out of solution).

Particle size reduction is another physical method that was mentioned which may be tried in order to increase a compound's solubility. Particle size reduction is reported to increase the surface area of the solid phase that is in contact with the liquid medium. However, particle size reduction cannot alter the solubility of the compound in a solvent, which is a thermodynamic quantity. It may compensate for a slow rate of dissolution (a kinetic phenomenon) by increasing the amount of solid compound that is available to “react” with the solvent, i.e. to dissolve. Consequently, when a compound is known to have very poor solubility in a liquid, a decrease in particle size alone cannot be predicted to improve solubility because particle size reduction is reported not to affect thermodynamic stability. There is no suggestion in the art that the low solubility of lamotrigine is attributable to anything other than its low intrinsic, thermodynamic solubility in water.

U.S. Pat. No. 5,861,179 (the '179 patent) discloses a solid pharmaceutical formulation comprising lamotrigine. The formulation is made of granules having a particle size of 850 μm or less. The granules are prepared by spray granulating lamotrigine or lamotrigine salt with lactose, starch and crystalline cellulose in the presence of polyvinylpyrrolidone as binder. The '179 patent states that the active ingredient and excipients have particle sizes below 200 μm before granulation. The lamotrigine or lamotrigine salt employed as a starting material typically has a particle size of 125 μm or less.

There is a long-felt and unmet need in the art of pharmacology and especially in the field of parenteral formulations for lamotrigine.

### SUMMARY OF THE INVENTION

The present invention provides a plurality of lamotrigine particles having a specific surface area of from about two to about three and a half square meters per gram. More preferably, the lamotrigine particles having a specific surface area of about three square meters per gram.

The present invention provides a plurality of lamotrigine particles having the diameter of all particles in the plurality is equal to or less than about 100 μm; preferably, is equal to or less than about 50 μm; and most preferably, is equal to or less than about 10 μm.

The present invention provides a pharmaceutical composition comprising a plurality of lamotrigine particles having a specific surface area of from about two to about three and a half square meters per gram. More preferably, the lamotrigine particles having a specific surface area of about three square meters per gram.

The present invention provides a pharmaceutical composition of a plurality of lamotrigine particles. The lamotrigine particles have the diameter of all particles in the plurality is equal to or less than about 100 μm; preferably, is equal to or less than about 50 μm; and most preferably, is equal to or less than about 10 μm.

The pharmaceutical composition can be formulated into a wide variety of dosage forms including being used to prepare solutions of lamotrigine for oral or parenteral administration. Preferably, the dosage form is a solid oral dosage, a liquid oral dosage or a liquid parenteral dosage.

The present invention provides a dosage form comprising the pharmaceutical composition of lamotrigine of defined morphology. Preferably, the dosage form is a solid oral dosage form comprising at least one pharmaceutically acceptable excipient.

Preferably, the solid oral dosage form containing a unit dose of from about 100 to about 400 milligrams of lamotrigine.

The present invention provides a liquid oral dosage form comprising a liquid carrier selected from the group consisting of water, vegetable oil, alcohol, polyethylene glycol, propylene glycol and glycerin.

The present invention provides a liquid dosage form of further comprising at least one excipient. Preferably, the liquid parenteral dosage form further comprising a tonicity modifier. More preferably, the tonicity modifier is dextrose. More preferably, the aqueous vehicle and tonicity modifier is a 5% solution of dextrose.

The present invention provides a liquid parenteral dosage form of lamotrigine of defined morphology further comprising at least one excipient selected from the group consisting of dextrose, glycerol, lactose, mannitol, sorbitol, acetate, citrate, tartrate, parabens, 1,6-dialkyl substituted phenols, benzalkonium chloride, benzethonium chloride, benzyl alcohol, sodium benzoate, chlorobutanol, phenethyl alcohol, sodium bisulfite, sodium metabisulfite and tocopherol.

The present invention provides a method of reducing the incidence of seizures in a patient comprising the step of administering the dosage forms of lamotrigine of defined morphology.

The present invention further provides the lamotrigine dosage form to be administered in adjunct with another seizure inhibiting drug.

### DESCRIPTION OF THE PREFERRED EMBODIMENTS

The present invention provides a plurality of lamotrigine particles of defined morphology. “Defined morphology” of the lamotrigine particles is characterized by their specific surface area and particle diameter. In accordance with the invention, the plurality of lamotrigine particles is characterized by having a specific surface area of from about 2.0 to about 3.5 square meters per gram. More preferably, the specific surface area is about 3 square meters per gram. The diameter of all lamotrigine particles in the plurality is equal to or less than about 100 μm; preferably, is equal to or less than about 50 μm; and most preferably, is equal to or less than about 10 μm.

The present invention also provides a pharmaceutical composition comprising a plurality of lamotrigine particles characterized by the specific surface area and particle diameter. The pharmaceutical composition is useful for preparing compressed solid dosage forms, encapsulated free flowing and compressed dosage forms, enteral solutions, suspensions and elixers and parenteral solutions.

The pharmaceutical composition of this invention comprises a plurality of lamotrigine particles. Particles of the plurality will vary in characteristics and the characteristics of no individual or small proportion of the particles will materially affect the advantages afforded by this invention which may include more rapid dissolution and the potential for improved bioavailability. Rather, the characteristics of the pharmaceutical composition are determined from a statistically significant sampling of the composition and measurement of bulk, or average, properties of the sample. Statistically significant measurements include those with a statistical sampling error of about 2% or less.

A “pharmaceutical composition,” as used herein, means a medicament for use in treating a mammal that comprises lamotrigine prepared in a manner that is appropriate for administration to a mammal; preferably, a human. A pharmaceutical composition also may contain one or more pharmaceutical excipients that are non-toxic to the mammal intended to be treated when the composition is administered in an amount effective to treat the mammal. A pharmaceutical composition includes feedstocks for preparing pharmaceutical dosage forms such as tablets, capsules, suspensions or solutions.

The “particle diameter” refers to the equivalent spherical diameter as determined by laser light scattering method.

Unless otherwise specified, the term “about” has the meaning given to it by those well acquainted with the arts to which this invention pertains and where not in conflict with the understanding of skilled artisans, by its customary and accepted meaning.

“Specific surface area” is the ratio of the surface area of lamotrigine particles to its unit mass; expressed herein in square meters/gram. The present invention also provides a pharmaceutical formulation comprising a plurality of lamotrigine particles having a specific surface area of from about 2.0 to about 3.5 square meters per gram. More preferably, the specific surface area is about 3 square meters per gram.

Gas adsorption analysis is a preferred method for determining the surface area of a sample of lamotrigine for purposes of this invention since it provides a direct measurement of surface area. It is therefore considered to be more accurate than other methods such as electron microscopy and laser diffraction that rely upon a model to obtain a value for surface area (e.g. equivalent sphere model).

We measured specific particle surface area by gas adsorption using a Coulter™ SA3100 Series adsorption analyzer. The specific surface area of a sample of lamotrigine particles was calculated by the multipoint Brunauer, Emmet and Teller (BET) method. Brunauer, S., P. H. Emmett, E. Teller, J. Am. Chem. Soc., 1938, 60, 309-319. The calculations of the surface area of lamotrigine particles and its precise weight would yield specific surface area (usually expressed as square meters/gram). Accurate determination of the specific surface area of the sample depends upon proper sample preparation. Pure samples should be weighed to ±0.001 gram and thoroughly desorbed of adsorbed gases before analysis. This may be done by exposing the sample to high dynamic vacuum (≦0.0001 torr) at about 70° C. for thirty minutes. An approximately one gram sample was loaded into a sample tube (9 cc) of the Coulter gas adsorption analyzer and thoroughly degassed. The volume of the manifold was measured with helium gas using a conventional technique. Adsorption data was generated at −77° C. using nitrogen gas (P/P₀ N₂=0.05-0.3) as adsorbate. The instrument's software calculated the moles of adsorbed N₂ (based upon inputted sample mass, manifold volume and sample tube void volume) for ten equilibrium pressures (Pₛ). In this range, linearity of the BET equation did not deviate significantly (r=0.999).

The number of moles of N₂ adsorbed in a monolayer (nₘₒₙₒ) onto the surfaces of the lamotrigine particles was calculated by application of the least squares method to the BET equation in its linear form y=ax+b wherein the variables y=Pₛ/n(P₀-Pₛ) and x=(Pₛ/P₀) in which n is the number of moles of N₂ adsorbed and condensed onto the sample, nₘₒₙₒ=1/(a+b) and the other variables are as previously defined. The specific surface area of the particles was then routinely calculated.

Pharmaceutical compositions of the invention include those whose lamotrigine particles meet the invention's surface area criteria and whose lamotrigine particles have a particle diameter of 100 μm or less, more preferably about 50 μm or less and most preferably about 10 μm or less. Lamotrigine having these specific surface areas is advantageous for solubility, bioavailability and rate of dissolution.

Yet more preferred pharmaceutical compositions of the invention contain no lamotrigine particles of diameter greater than about 100 μm.

Lamotrigine of defined particle size may be produced by precipitation from appropriate solvents. Particle diameter may be adjusted by customary methods such as cooling, pH adjustment, pouring a concentrated solution into an anti-solvent and/or by co-precipitation so as to obtain a precipitate with the appropriate specific surface area.

Lamotrigine of defined particle diameter may be produced by known methods of particle size reduction starting with crystals, powder aggregates and course powder of either crystalline or amorphous lamotrigine. The principal operations of conventional size reduction are milling of a feedstock material and sorting of the milled material by size.

A fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid (typically air) stream to cleave the particles. The suspended particles are injected under pressure into a recirculating particle stream. Smaller particles are carried aloft inside the mill and swept into a vent connected to a dust collector. The feedstock may be pre-milled to about 150 to 850 μm.

The most widely practiced method of sorting by particle size involves passing the milled material through a stack of sieves, each with openings of a different size. The sieves are arranged so that the material encounters the sieve having the largest openings first and those particles that pass through the first sieve encounter a second sieve with smaller openings and those that pass through the second sieve may encounter a third sieve, and so forth. Lamotrigine particles also may be separated by particle size using cyclonic or centrifugation techniques.

Sieves are constructed of wire cloth or finely perforated sheet mounted in a frame. Sieves are classified by the size openings in the wire cloth or perforated sheet. The size of the openings in a wire cloth sieve is determined by the diameter of the wire and the tightness of the weave of the wire cloth. The ASTM E11-01 (available through the worldwide web at http://www.astm.org) series of Standard Test Sieves include sieves with 106 μm, 53 μm and 32 μm openings. Particles with a diameter of from about 100 μm to about 50 μm can be separated from a powder with a broader particle size distribution by using a stack of two-sieves with a 106 μm seive above a 53 μm seive. When the material encounters the sieve stack, particles larger than 106 μm are captured on the first sieve. The oversized particles can be remilled to produce more particles within the desired size range. Fines of diameter less than 53 μm would be allowed to pass through the second sieve and also may be recycled, such as by dissolving them and recrystallizing or granulating them to produce a feedstock suitable for size reduction. Particles within the about 100-50 size range would be collected on the second, 53 μm sieve.

Industry has developed different systems for designating sieve sizes. For instance, 106 μm, 53 μm and 32 μm sieves of the ASTM Standard designation system correspond to Nos. 140, 270 and 450 respectively of an alternative ASTM designation system based on the English system. Another distinct system whose designations parallel those of the ASTM Alternative designation system is the Tyler Designation. The Tyler system is sometimes intended to be referred to when a sieve's size is given in “mesh.” One hundred and six micrometer (ASTM Alt. No. 140) and 32 μm (ASTM Alt. No. 270) sieves are designated 150 mesh and 270 mesh respectively in the Tyler system. See, Perry's Chemical Engineers' Handbook, 6th ed. p. 21-15 (1984). This disclosure uses the ASTM E11-01 Standard Test Sieve designation.

The pharmaceutical composition of this invention may be formulated into a variety of solid and liquid dosage forms for administration to humans and/or animals. The dosage forms include those suitable for enteral (oral, buccal, rectal) administration and parenteral (including subcutaneous, intramuscular, and intravenous) administration. Dosage forms include solid dosage forms like tablets, powders, capsules, suppositories, sachets, troches and losenges as well as liquid solutions, suspensions, syrups and elixirs. The most suitable route in any given case will depend on the nature and severity of the condition being treated and other circumstances that will be assessed by the caregiver.

The pharmaceutical composition may be made into a solid oral dosage form such as a tablet. For making a tablet, it will typically be desirable to include one or more benign pharmaceutical excipients (i.e., pharmaceutical acceptable excipients) in the composition. The pharmaceutical composition of the present invention may contain one or more diluents added to make the tablet larger and, hence, easier for the patient and caregiver to handle. Common diluents are microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Binders also may be included to help hold the tablet together after compression. Some typical binders are acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The tablet may further include a disintegrant to accelerate disintegration of the tablet in the patient=s stomach. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

A pharmaceutical composition for tableting may further include glidants, lubricants, flavorings, colorants and other commonly used excipients.

Liquid oral dosage form of the present invention comprise lamotrigine of defined morphology and a liquid carrier such as water, vegetable oil, alcohol, polyethylene glycol, propylene glycol or glycerin, most preferably water.

Liquid oral dosage form may contain emulsifying agents to disperse uniformly throughout the composition the active ingredient or other excipient that has low solubility in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid oral dosage form of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

The liquid oral dosage form also may contain sweetening agents, such as sorbitol, saccharin, sodium saccharin, sucrose, aspartame, fructose, mannitol and invert sugar; preservatives and chelating agents such as alcohol, sodium benzoate, butylated hydroxy toluene, butylated hydroxyanisole and ethylenediamine tetraacetic acid; and buffers such as gluconic acid, lactic acid, citric acid or acetic acid, sodium gluconate, sodium lactate, sodium citrate or sodium acetate.

A preferred dosage form of lamotrigine is a pressurized container of the pharmaceutical composition of the invention that is suitable for the administering lamotrigine as an inhalant.

A preferred dosage form of lamotrigine is a sterile solution for parenteral (e.g., intravenous) administration comprising an aqueous vehicle and lamotrigine, wherein the solution is prepared by contacting a pharmaceutical composition in accordance with this invention with a sterile aqueous liquid. The sterile solution may further contain a tonicity modifying agent such as dextrose glycerol, lactose, mannitol, sorbitol and the like to establish and maintain a suitable osmotic pressure in the aqueous phase. An especially preferred aqueous vehicle with tonicity modifier is a 5% aqueous dextrose solution. The solution for injection may further include a buffer such as acetate, citrate and tartrate; an antimicrobial agent such as parabens, 1,6-dialkyl substituted phenols, benzyl alcohol, sodium benzoate, chlorobutanol, phenethyl alcohol and the like; antioxidants like sodium bisulfite, sodium metabisulfite and tocopherol and the like; as well as any conventional additives that do not cause significant decomposition, hydrolysis of the lamotrigine or ion exchange leading to an insoluble precipitate. For the latter purpose, the use of chloride salts and phosphates is in a parenteral formulation is discouraged. Intravenous administration will be by a series of injections or by continuous infusion over an extended period. Administration by injection or other routes of discretely spaced administration will generally be performed at intervals ranging from weekly to once to three times daily.

A preferred unit dose is a vial or septum-sealed bottle containing the injectable solution of the invention. For adjunctive therapy involving oral administration of a solid or liquid dosage form, an orally administered unit dosage contains normally from 0.15 mg kg⁻¹ day⁻¹ to 25 mg kg⁻¹ day⁻¹. The dosage is generally from 100-400 mg day⁻¹ for adult humans given in single or divided does.

Having thus described the present invention with reference to certain preferred embodiments, the present invention is further illustrated by the following example. The example is provided for illustrative purposes only and is not intended to limit in any way the invention which is defined by the claims.

### EXAMPLE 1

Initially, the lamotrigine particles had a particle diameter distribution wherein 50% of the particles had a diameter equal to or less than 250 μm, and about 80% of the particles had a diameter equal to or less than about 500 μm.

These particles were fed into a 300 mm horizontal type air jet mill operating under the following parameters:
Air supplied: Oil free and dried to a dew point of less than 3° C.
Air flow rate: 7 Nm³/min.
Air pressure: 10 bar
Feed air pressure: 6 bar
Grinding air pressure: 4 bar
Nozzle angle: 32.05°
Feed rate: 29 Kg/h

Particle diameter of lamotrigine was determined by laser diffraction. We determined the diameter of lamotrigine particles using a Malvern™ Mastersizer laser diffraction instrument. Samples of lamotrigine were suspended in hexane containing a surfactant (0.07% dioctyl sulfosuccinate sodium salt). The suspensions were mixed and then sonicated for 15 seconds to thoroughly disperse the lamotrigine particles. The dispersion was then recirculated in the flow cell of the Malvern Mastersizer for two minutes before particle diameter measurements were taken.

The micronized product contained a plurality of lamotrigine particles and the diameter of all particles in the plurality was less than 100 μm.

### EXAMPLE 2

The specific surface area of the lamotrigine particles was measured with a Coulter SA 3100, using the multipoint BET technique. About 1 gram of powder was introduced and weighted in a 9 c.c. tube. The experimental parameters of degassing were 70° C., 30 minutes. For the linearity, 10 points were measured, using high sensitivity.

The value of specific surface area for lamotrigine micronized particles was measured to be 3.1-3.3 square meters per gram.

Having thus described the invention with respect to certain preferred embodiments and further illustrated it with the example, those skilled in the art may come to appreciate substitutions and equivalents that albeit not expressly described are taught and inspired by this invention. Whereas such substitutions and equivalents do not depart from the spirit of the invention they are within its scope which is defined by the claims which follow.

1. A plurality of lamotrigine particles having a specific surface area of from about two to about three and a half square meters per gram.

2. The plurality of lamotrigine particles of claim 1 having a specific surface area of about three square meters per gram.

3. The plurality of lamotrigine particles of claim 1 wherein the diameter of all particles in the plurality is equal to or less than about 100 μm.

4. The plurality of lamotrigine particles of claim 3 wherein the diameter of all particles in the plurality is equal to or less than about 50 μm.

5. The plurality of lamotrigine particles of claim 4 wherein the diameter of all particles in the plurality is equal to or less than about 10 μm.

6. A pharmaceutical composition comprising a plurality of lamotrigine particles having a specific surface area of from about two to about three and a half square meters per gram.

7. The pharmaceutical composition of claim 6 having a specific surface area of about three square meters per gram.

8. The pharmaceutical composition of claim 6 wherein the diameter of all particles in the plurality is equal to or less than about 100 μm.

9. The pharmaceutical composition of claim 8 wherein the diameter of all particles in the plurality is equal to or less than about 50 μm.

10. The pharmaceutical composition of claim 9 wherein the diameter of all particles in the plurality is equal to or less than about 10 μm.

11. A dosage form comprising the pharmaceutical composition of claim 6.

12. The dosage form of claim 11 that is a solid oral dosage.

13. The solid oral dosage of claim 12 wherein the pharmaceutical composition comprises at least one pharmaceutically acceptable excipient.

14. The solid oral dosage form of claim 13 wherein the pharmaceutically acceptable excipient is selected from the group consisting of microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylate, potassium chloride, powdered cellulose, sodium chloride, sorbitol, talc, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methylcellulose, liquid glucose, magnesium aluminum silicate, maltodextrin, methylcellulose, polymethacrylates, povidone, pregelatinized starch, sodium alginate, starch, alginic acid, carboxymethyl cellulose calcium, colloidal silicon dioxide, croscarmellose sodium, crospovidone, guar gum, magnesium aluminum silicate, methyl cellulose, polacrilin potassium, powdered cellulose, pregelatinized starch, sodium alginate and sodium starch glycolate.

15. The solid oral dosage form of claim 12 containing a unit dose of from about 100 to about 400 milligrams of lamotrigine.

16. The dosage form of claim 11 that is a liquid oral dosage.

17. The liquid oral dosage of claim 16 wherein the liquid oral dosage comprises a liquid carrier selected from the group consisting of water, vegetable oil, alcohol, polyethylene glycol, propylene glycol and glycerin.

18. The liquid oral dosage of claim 17 wherein the liquid carrier is water.

19. The liquid oral dosage of claim 16 further comprising at least one excipient selected from the group consisting of gelatin, egg yolk, casein, cholesterol, acacia, tragacanth, chondrus, pectin, methyl cellulose, carbomer, cetostearyl alcohol, cetyl alcohol, alginic acid bentonite, carbomer, carboxymethylcellulose calcium or sodium, ethylcellulose, gelatin guar gum, hydroxyethyl cellulose, hydroxypropyl cellulose, hydroxypropyl methylcellulose, maltodextrin, polyvinyl alcohol, povidone, propylene carbonate, propylene glycol alginate, sodium alginate, sodium starch glycolate, starch tragacanth, xanthan gum, sorbitol, saccharin, sodium saccharin, sucrose, aspartame, fructose, mannitol, invert sugar; ethyl alcohol, sodium benzoate, butylated hydroxy toluene, butylated hydroxyanisole, ethylenediamine tetraacetic acid, guconic acid, lactic acid, citric acid, acetic acid, sodium guconate, sodium lactate, sodium citrate and sodium acetate.

20. The dosage form of claim 11 that is a liquid parenteral dosage.

21. The liquid parenteral dosage of claim 20 further comprising a tonicity modifier.

22. The liquid parenteral dosage of claim 21 wherein the tonicity modifier is dextrose.

23. The liquid parenteral dosage of claim 22 wherein the dextrose is a 5% solution of dextrose.

24. The liquid parenteral dosage of claim 20 further comprising at least one excipient selected from the group consisting of dextrose, glycerol, lactose, mannitol, sorbitol, acetate, citrate, tartrate, parabens, 1,6-dialkyl substituted phenols, benzalkonium chloride, benzethonium chloride, benzyl alcohol, sodium benzoate, chlorobutanol, phenethyl alcohol, sodium bisulfite, sodium metabisulfite and tocopherol.

25. A method of reducing the incidence of seizures in a patient comprising the step of administering a dosage form of any of claims 12, 16 and 20.

26. The method of claim 25 wherein the dosage form is administered in adjunct with another seizure inhibiting drug.
